(19) United States
(12) Patent Application Publication
(10) Pub. No.: US 2024/0316255 A1
(43) Pub. Date: Sep. 26, 2024

(54) METHOD AND SYSTEM FOR AUTOMATICALLY CHARACTERIZING A VASCULAR ACCESS OF A DIALYSIS PATIENT

(71) Applicant: B. Braun Avitum AG, Melsungen (DE)

(72) Inventors: Waldemar Janik, Melsungen (DE); Silvie Krause, Melsungen (DE)

(21) Appl. No.: 18/597,078

(22) Filed: Mar. 6, 2024

(30) Foreign Application Priority Data
Mar. 7, 2023 (DE) 10 2023 105 664.6

(51) Int. Cl. A61M 1/36; A61M 1/16

(52) U.S. Cl. CPC A61M 1/3658; A61M 1/1603; A61M 2205/3368; A61M 2205/3375; A61M 2205/50; A61M 2230/207

(57) ABSTRACT

A method automatically characterizes a vascular access of a dialysis patient who is or was connected to a dialysis machine, the vascular access being characterized dependent on a recirculation rate.

### CROSS-REFERENCE TO RELATED APPLICATION

This application claims priority under 35 U.S.C. § 119 to German Application No. 10 2023 105 664.6, filed on Mar. 7, 2023, the content of which is incorporated by reference herein in its entirety.

### FIELD

The present disclosure relates a method and a system for automatically characterizing a vascular access of a dialysis patient.

### BACKGROUND

Within the scope of dialysis, a shunt vessel for establishing a vascular access or shunt is generally punctured twice several times a week, whereby calcified constrictions or stenoses and wall sacs or aneurysms may form and excessive growth of the tunica intima (intimal hyperplasia) may be provoked. Over time, these changes may lead to a reduced volumetric flow rate through the access, i.e. a reduced shunt flow.

### SUMMARY

The present disclosure is based on the object of providing a method and a system for automatically characterizing a vascular access of a dialysis patient, by means of which a no longer sufficiently functional vascular access is rendered automatically detectable.

The method according to the present disclosure serves to automatically characterize a vascular access of a dialysis patient who is connected, or was connected at least once, to a dialysis machine. As is conventional, the vascular access in this case comprises an arterial part or access and a venous part or access. The vascular access is also referred to as a shunt. In this respect, reference is also made to the relevant literature in the art.

According to the present disclosure, the vascular access is characterized dependent on a recirculation rate. In this case, the recirculation rate R may for example be defined as the recirculation flow QR in relation to the extracorporeal blood flow QB, i.e. the following may apply:

$$R [ \% ] = ( QR / QB ) * 100 \% .$$

The recirculation rate typically depends on the blood flow. Since the blood flow is generally known, this dependence can be considered during the characterization, for example by virtue of the recirculation rate being normalized to the blood flow and the vascular access being characterized dependent on the recirculation rate, which has been normalized to the blood flow.

For example, the vascular access can be characterized quantitatively by way of a statistic, with higher numerical values of the statistic for example representing a functionally better vascular access. In a further example, the vascular access can be characterized qualitatively according to different grades, for example “very good”, “good”, “satisfactory”, “sufficient”, and “requiring revision”.

According to an embodiment, the vascular access is characterized as having a decreasing quality as the recirculation rate increases.

According to an embodiment, the recirculation rate is determined quantitatively or qualitatively by means of the following steps: changing at least one operational parameter of the dialysis machine, measuring a change in at least one blood value at the arterial access of the dialysis machine, and determining the recirculation rate dependent on the measured change in the at least one blood value at the arterial access of the dialysis machine.

According to an embodiment, the recirculation rate is determined as having greater values as a measured change increases. The higher the recirculation rate, the greater the effect of the change in the operational parameter on the blood value or values measured at the arterial access. As a consequence, the change in the measured blood value or values is characteristic for the recirculation rate.

According to an embodiment, the at least one operational parameter influences a change of a dialysate flow through the dialyzer of the dialysis machine.

The dialysis machine may comprise a sensor system at the dialysate outlet, for example an optical sensor or a conductivity probe.

Two successive bypasses for changing the flow through the dialyzer to a value of virtually 0 ml/min may be brought about for different time durations, with the resultant measurement signals from the sensor system being evaluated. The signal might be afflicted by recirculation after a short bypass. The signal is recirculation-free after a long bypass. Thus, a deviation between the two signals indicates a recirculation. The extent of the deviation depends on the recirculation rate.

According to an embodiment, the at least one operational parameter of the dialysis machine influences an ultrafiltration rate, and the at least one blood value contains or is a hematocrit value.

According to an embodiment, the ultrafiltration rate is increased, and the recirculation rate is determined dependent on an increase in the hematocrit value.

For example, in order to measure the hematocrit value, the dialysis machine may comprise a hematocrit sensor at its arterial access or tube portion. Optionally, a further hematocrit sensor may be provided at the venous access or tube portion. For example, the ultrafiltration rate may be increased briefly. This brings about an increased withdrawal of water and hence an increase in the hematocrit value in the venous blood. If recirculation is present, the recirculated blood thus thickened passes the hematocrit sensor at the arterial access, whereby an elevated hematocrit value is rendered measurable. This is sufficient for a qualitative statement as to whether or not recirculation is present. For a quantitative statement, the hematocrit value can be additionally measured at the venous access. The ratio of venous to arterial hematocrit value or a ratio of associated signal areas correlates with the recirculation rate level.

According to an embodiment, the at least one operational parameter influences a change in the temperature of the outflowing blood at the venous access of the dialysis machine, and the at least one blood value contains or is a temperature of the blood flowing through the arterial access of the dialysis machine.

For example, the dialysis machine may comprise a temperature-change adjustment unit at the venous tube or access. Alternatively, a change in temperature may also be brought about by the addition of a fluid volume (e.g. NaCl solution) which is colder or warmer in comparison with the blood. An optional temperature sensor may be provided downstream in the direction of blood flow. Further, a temperature sensor is provided at the arterial tube or access.

The blood temperature can be changed briefly at the venous access. In the case of a recirculation, the change in temperature would also be measurable in the arterial access or tube. This is sufficient for a qualitative statement as to whether or not recirculation is present. For a quantitative statement, the temperature can be additionally measured at the venous portion or access. The ratio of venous to arterial temperature or the ratio of the two areas under the temperature curves correlates with the recirculation rate level.

According to an embodiment, the at least one operational parameter influences a temperature of a dialysis fluid, and the at least one blood value contains or is a temperature of the blood flowing through the arterial access of the dialysis machine.

The dialysis machine may comprise a heating element controlling the temperature of the dialysis fluid. Further, a temperature sensor may be provided at the arterial access or tube, and optionally additionally at the venous tube or access. Further, a temperature sensor may be optionally provided, i.e. not additionally provided, at the dialysate outlet of the dialyzer.

The temperature of the dialysis fluid can be changed briefly. This change in temperature is transmitted in the dialyzer to the blood flowing therethrough. In the case of a recirculation, the change in temperature would also be measurable in the arterial access or tube. This is sufficient for a qualitative statement as to whether or not recirculation is present. For a quantitative statement, the temperature can be additionally measured at the venous portion or access. The ratio of venous to arterial temperature or the ratio of the two areas under the temperature curves correlates with the recirculation rate level.

With a time delay, the temperature bolus within the dialyzer can pass from the blood side back to the dialysate side, with the result that a temperature change would likewise be measurable at the dialysate outlet if a recirculation is present.

According to an embodiment, the at least one operational parameter influences an addition of a substance, in particular NaCl, into the outflowing blood at the venous access of the dialysis machine, and the at least one blood value contains or is a concentration of the substance in the blood flowing through the arterial access of the dialysis machine.

For example, the blood value can also be an arterial pressure in the case of a stationary blood pump while the access is established.

According to an embodiment, the concentration of the substance is measured on the basis of a speed of sound in the blood flowing through the arterial access of the dialysis machine.

The dialysis machine can comprise an apparatus for measuring the speed of sound at the arterial access or tube and optionally at the venous access or tube.

For example, an NaCl solution can be added to the venous blood flowing back to the patient. Since blood and NaCl solution have different densities, a change in the speed of sound would be measurable at the arterial sensor if recirculation is present.

The bolus can be added manually or automatically if the dialysis machine and the tube system comprise appropriate apparatuses for administering a dialysis fluid bolus.

According to an embodiment, the recirculation rate is ascertained quantitatively or qualitatively on the basis of a comparison between a measured clearance and a nominal clearance for the dialysis machine.

For example, the dialysis machine may comprise an apparatus for determining clearance, for example in the form of an optical sensor at the dialysate outlet or a conductivity probe at the dialysis fluid inlet and a probe at the dialysate outlet. Further alternative sensors for determining the dialysate composition (e.g. ion concentration) are conceivable. Further, a blood-side pressure sensor may be provided at the blood inlet of the dialyzer.

Theoretically obtainable performance parameters of a dialyzer are known, for example stored in a table, or determinable. In particular, this includes the clearance. The clearance may deviate if what is known as a secondary membrane is formed in the dialyzer. However, this secondary membrane would also lead to an increase in the blood inlet pressure and would consequently be identifiable. However, the clearance also depends on the recirculation rate. If the blood inlet pressure is in the normal range, i.e. no secondary membrane is present, a reduced clearance may indicate a recirculation.

Clearance can be determined intradialytically in different ways.

For example, the composition of the dialysis fluid can be changed briefly, and the conductivities at the dialysis fluid inlet and dialysate outlet can be measured before and after the change.

Further, a bypass may be established, i.e. the dialysis fluid can briefly be guided past the dialyzer, until a diffusive equilibrium between the blood and dialysate sides is established in the dialyzer. A signal extremum can subsequently be measured at the dialysate outlet. This corresponds to the blood inlet value, and so the clearance can subsequently be determined according to equations known per se.

Theoretically obtainable and practically obtained clearance values can be compared with one another, with the blood inlet pressure being taken into account. A deviation between the values indicates recirculation.

Clearance can also be determined by brief changes in the blood or dialysate flow if for example the conductivity is measured on the dialyzer inlet and outlet side.

Each of the above-described method enables a qualitative and/or quantitative determination of the recirculation rate. Combinations of the described variants are also conceivable. Accurate figures for the recirculation rate are not mandatory. In addition to these point values of the respective recirculation rate in time, it is also possible to evaluate trends, i.e. the development of the values over time, for the purpose of characterizing the vascular access. For example, one possible evaluable parameter would be the slope of a regression line over a defined period of time comprising at least two measurements.

According to an embodiment, the method also includes the following steps: ascertaining the recirculation rate by means of the dialysis machine or by means of another piece of measurement equipment, transmitting the ascertained recirculation rate to a central processing unit via a data network, transmitting further data characteristic for the quality of the vascular access to the central processing unit via the data network, and characterizing the vascular access by means of the central processing unit on the basis of the recirculation rate and the further data.

According to an embodiment, the further data are selected from a set of data. The set of data contains at least one of the following data elements: a volumetric flow rate flowing through the access, a Kt/V value, in particular obtained on the basis of an analysis of pre- and/or post-dialytic blood samples of the dialysis patient, dialysis patient-related data, in particular in the form of the height, age, weight and/or sex of the dialysis patient, a date the vascular access was established, and a date of a preceding vascular access revision or shunt revision.

According to an embodiment, the recirculation rate and the further data are evaluated by means of machine learning or what is known as artificial intelligence for the purpose of characterizing the vascular access. On the basis of patient-related data, it is possible to use only data for learning which are similar to the patient for whom a prognosis should be made. It may be selectable whether the prognosis should be made on the basis of a model from all data or a model based on a similarity subset data model.

According to an embodiment, an alert is output, for example in the form of an acoustic warning signal, a visual warning signal, a haptic warning signal, etc., dependent on the characterization of the vascular access, in particular if the vascular access is characterized as insufficient.

The system according to the present disclosure for automatically characterizing a vascular access of a dialysis patient who is or was connected to a dialysis machine comprises: a dialysis machine and a central processing unit, the dialysis machine and the central processing unit each being designed to carry out an above-described method.

### BRIEF DESCRIPTION OF THE DRAWINGS

The present disclosure is described in detail below with reference to the drawings. In this case, very schematically:

FIG. 1 shows a system for automatically characterizing a vascular access of a dialysis patient in an initial state, and

FIG. 2 shows a relevant section of the system of FIG. 1 in a subsequent state.

### DETAILED DESCRIPTION

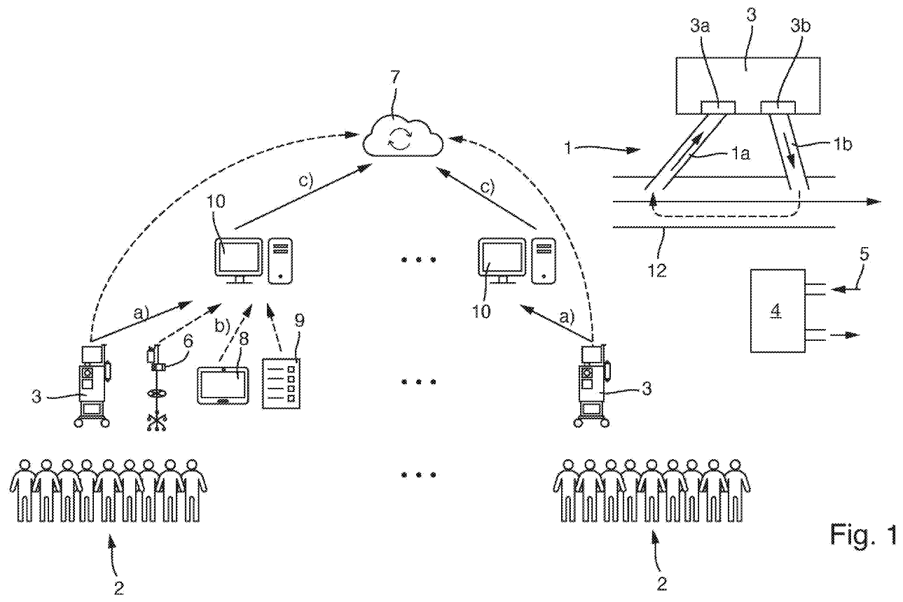

FIG. 1 very schematically shows a system for automatically characterizing a vascular access 1 of a dialysis patient 2 who is or was connected to a dialysis machine 3.

Conventionally, the vascular access into a vessel 12 of the patient 2 comprises an arterial part 1a and a venous part 1b, with the arterial part 1a being fluid-connected to a corresponding arterial access 3a of the dialysis machine 3 and the venous part 1b being fluid-connected to a corresponding venous access 3b of the dialysis machine 3.

The system comprises: the dialysis machine 3 having a dialyzer 4, an optional piece of measurement equipment 6, for example for measuring the recirculation rate and measuring the shunt flow, a central processing unit 7, components 8 and 9 for patient data management and a data management system 10.

Components 3, 6, 7, 8, 9 and 10 are data-connected to one another.

Components 3, 6, 7, 8 and 9 can be present multiple times as a component group, as illustrated.

The automatic characterization of the vascular access 1 of the dialysis patient 2 is implemented as described hereinbelow.

First, the dialysis machine 3 determines the recirculation rate. The result is sent to the data management system (DMS) 10, as symbolized by reference sign a).

Additionally, further data, such as a recirculation rate measurable by means of the measurement equipment 6 or a shunt flow measurable by means of the measurement equipment 6, can be sent to the data management system 10, as symbolized by reference sign b).

Further, data, for example in the form of a Kt/V value or a Kt/V curve, can be sent to the data management system 10, as likewise symbolized by reference sign b). For example, the Kt/V value can be based on the measurement of pre- and post-dialytic blood samples in a laboratory.

Finally, patient-related data, such as e.g. height, age, weight, sex, date of vascular access establishment, date of the last vascular access revision, etc., stored in components 8 and 9 for patient data management can be sent to the data management system 10, as likewise symbolized by reference sign b). For example, these data may originate from a (tablet) computer 8 or be transmitted manually to the data management system 10. Further, a date of establishment, a type of access, particular events regarding treatments (enterable by the patient or medical staff, for example in the form of secondary hemorrhages), etc. can be sent to the data management system 10.

The data management system 10 transmits the data to the central processing unit 7 via a data network, as symbolized by reference sign c). The processing unit 7 is in what is known as a cloud. In the cloud, all data are processed by the central processing unit 7 on the basis of machine learning.

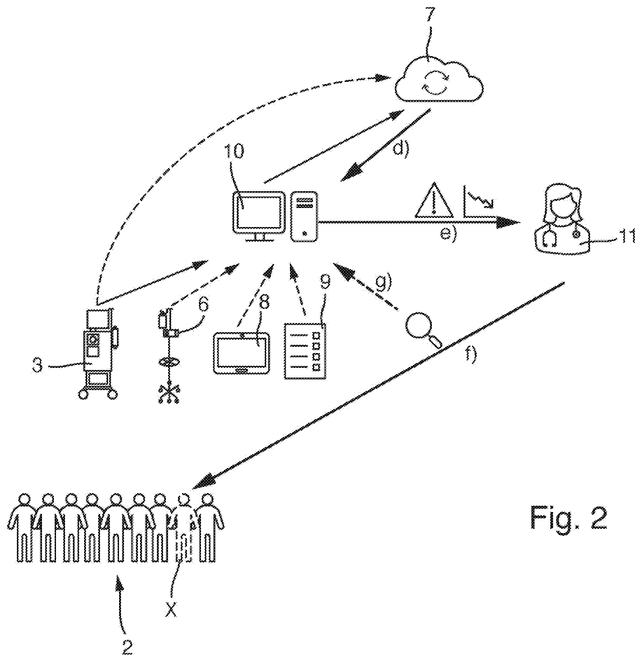

Referring now to FIG. 2, the central processing unit 7, after the data has been analyzed, reports to the data management system 10 that the vascular access 1 has deteriorated, or is in the process of deteriorating, in patient X, for example because the ascertained recirculation rate has increased over the past few weeks. This is symbolized by reference sign d).

As a consequence, the data management system 10 outputs an alert or a notification for medical staff 11, as symbolized by reference sign e).

The medical staff 11 examines the vessel, for example using a stethoscope, an imaging method, or a triggered shunt flow or recirculation measurement, as symbolized by reference sign f).

The result of this examination can be fed together with the other data to the central processing unit 7 via the data management system 10 in order to trigger an adaptation of the assessment algorithm implemented in the processing unit 7 by recursive learning in said processing unit, as symbolized by reference sign g).

Unlike the depiction, the data management system 10 can be a constituent part of the central processing unit 7, or the data management system 10 also adopts the aforementioned tasks of the central processing unit 7.

How the dialysis machine 3 is able to measure the recirculation rate will be described below on the basis of a few examples.

In principle, the vascular access 1 is characterized as having a decreasing quality as the recirculation rate increases.

At least one operational parameter of the dialysis machine 3 can be changed for the purpose of the quantitative or qualitative determination of the recirculation rate. Subsequently, a change in at least one blood value at the arterial access 3a of the dialysis machine 3 is measured, and the recirculation rate is ascertained dependent on the measured change in the at least one blood value at the arterial access 3a of the dialysis machine 3. The recirculation rate is ascertained as having greater values as a measured change increases.

The at least one operational parameter can influence a change of a dialysate flow through the dialyzer 4 of the dialysis machine 3. In an alternative or in addition, the at least one operational parameter of the dialysis machine 3 can influence an ultrafiltration rate, and the at least one blood value is a hematocrit value. For example, the ultrafiltration rate is increased, and the recirculation rate is determined dependent on an increase in the hematocrit value. Further, the at least one operational parameter can influence a change in the temperature of the outflowing blood at the venous access 3b of the dialysis machine 3, and the at least one blood value is a temperature of the blood flowing through the arterial access 3a of the dialysis machine. Further, the at least one operational parameter can influence a temperature of a dialysis fluid 5, and the at least one blood value is a temperature of the blood flowing through the arterial access 3a of the dialysis machine 3. Further, the at least one operational parameter can influence an addition of a substance, in particular NaCl, into the outflowing blood at the venous access 3b of the dialysis machine 3, and the at least one blood value is a concentration of the substance in the blood flowing through the arterial access 3b of the dialysis machine 3. For example, the concentration of the substance can be measured on the basis of a speed of sound in the blood flowing through the arterial access 3b of the dialysis machine 3.

Further, the recirculation rate can be ascertained quantitatively or qualitatively on the basis of a comparison between a measured clearance and a nominal clearance for the dialysis machine 3.

1. A method for automatically characterizing a vascular access of a dialysis patient who is or was connected to a dialysis machine, the method comprising the steps of:
determining a recirculation rate;
characterizing the vascular access based on the recirculation rate.

2. The method according to claim 1, wherein the vascular access is characterized as having a decreasing quality as the recirculation rate increases.

3. The method according to claim 1, wherein:
the recirculation rate is determined quantitatively or qualitatively by:
changing at least one operational parameter of the dialysis machine;
measuring a change in at least one blood value at an arterial access of the dialysis machine; and
determining the recirculation rate dependent on the change in the at least one blood value at the arterial access of the dialysis machine.

4. The method according to claim 3, wherein the recirculation rate is determined as having greater values as the change increases.

5. The method according to claim 3, wherein the at least one operational parameter influences a change of a dialysate flow through a dialyzer of the dialysis machine.

6. The method according to claim 3, wherein:
the at least one operational parameter of the dialysis machine influences an ultrafiltration rate; and
the at least one blood value contains a hematocrit value.

7. The method according to claim 6, wherein the ultrafiltration rate is increased, and the recirculation rate is determined dependent on an increase in the hematocrit value.

8. The method according to claim 3, wherein:
the at least one operational parameter influences a change in the temperature of outflowing blood at a venous access of the dialysis machine, and
the at least one blood value contains a temperature of blood flowing through the arterial access of the dialysis machine.

9. The method according to claim 3, wherein:
the at least one operational parameter influences a temperature of a dialysis fluid, and
the at least one blood value contains a temperature of blood flowing through the arterial access of the dialysis machine.

10. The method according to claim 3, wherein:
the at least one operational parameter influences an addition of a substance into outflowing blood at the venous access of the dialysis machine, and
the at least one blood value contains a concentration of the substance in the blood flowing through an arterial access of the dialysis machine.

11. The method according to claim 10, wherein the concentration of the substance is measured based on a speed of sound in blood flowing through the arterial access of the dialysis machine.

12. The method according to claim 1, wherein the recirculation rate is ascertained quantitatively or qualitatively based on a comparison between a measured clearance and a nominal clearance for the dialysis machine.

13. The method according to claim 1, further comprising the steps of:
ascertaining the recirculation rate with the dialysis machine or with another piece of measurement equipment;
transmitting the recirculation rate to a central processing unit;
transmitting further data characteristic of a quality of the vascular access to the central processing unit, and
characterizing the vascular access with the central processing unit based on the recirculation rate and said further data.

14. The method according to claim 13, wherein said further data are selected from a set of data containing at least one of the following data elements:
a volumetric flow rate flowing through the vascular access;
a Kt/V value;
dialysis patient-related data;
a date the vascular access was established; and
a date of a preceding vascular access revision.

15. The method according to claim 13, wherein the recirculation rate and the further data are evaluated by machine learning for characterizing the vascular access.

16. The method according to claim 1, wherein an alert is output dependent on a characterization of the vascular access.

17. A system for automatically characterizing a vascular access of a dialysis patient who is or was connected to a dialysis machine, comprising:
the dialysis machine; and
a central processing unit,
the dialysis machine and the central processing unit being designed to carry out a method according to claim 1.
